(19) United States
(12) Patent Application Publication
(10) Pub. No.: US 2024/0423491 A1
(43) Pub. Date: Dec. 26, 2024

(54) HEART RATE ESTIMATION FROM FACIAL IMAGES USING IMAGE PROCESSING AND SELF-ADAPTIVE MATRIX NOISE REMOVAL

(71) Applicant: Mohammadreza Tavasoli Naeini, Isfahan (IR)

(72) Inventor: Mohammadreza Tavasoli Naeini, Isfahan (IR)

(21) Appl. No.: 18/793,815

(22) Filed: Aug. 4, 2024

(51) Int. Cl. A61B 5/024; A61B 5/00; A61B 5/103; G06T 5/70; G06T 7/00; G06T 7/20; G06T 7/50; G06T 7/90; G06V 10/74

(52) U.S. Cl. CPC A61B 5/02427; A61B 5/1032; A61B 5/441; G06T 5/70; G06T 7/0012; G06T 7/20; G06T 7/50; G06T 7/90; G06V 10/761; G06T 2207/10016; G06T 2207/10024; G06T 2207/20004; G06T 2207/20072; G06T 2207/20084; G06T 2207/30088; G06T 2207/30201; G06V 2201/03

(57) ABSTRACT

The invention of heart rate estimation from facial images using image processing and self-adaptive matrix noise removal relates to heart rate detection through image processing and machine learning algorithms which involves the analysis of skin color changes and incorporates noise removal techniques by identifying the most reliable points and employing a self-adaptive matrix. This system employs a high-quality camera to capture video footage of a person's face and also through sophisticated image processing and machine vision methods, the system monitors and analyzes the subtle color changes in the facial skin caused by the contraction and expansion of blood vessels with each heartbeat.

### TECHNICAL FIELD OF THE INVENTION

The present invention pertains to the medical field, specifically to heart rate detection through image processing and machine learning algorithms. It involves the analysis of skin color changes and incorporates noise removal techniques by identifying the most reliable points and employing a self-adaptive matrix.

### PRIOR ARTS

There are various methods for heart rate estimation, ranging from simple to advanced techniques. One common method is manual measurement, which involves using the wrist or neck pulse. To do this, place two fingers (usually the index and middle fingers) on the wrist, count the pulses for 15 seconds, and then multiply the result by 4 to estimate the heart rate per minute. Additionally, electronic devices, such as heart rate monitors and echocardiography equipment, are widely used, including smartwatches and advanced medical equipment available in hospitals and clinics. Another approach involves imaging techniques, such as infrared imaging and photoplethysmography (PPG). Infrared imaging tracks changes in blood flow on the skin's surface using infrared cameras, while PPG utilizes optical sensors to measure variations in blood volume within body tissues through the emission and absorption of light. Furthermore, heart rate can be measured using techniques based on electromagnetic waves, such as electrocardiogramar EKG) and methods utilizing radio and radar waves. Moreover, audio techniques like stethoscopes and digital audio devices are commonly used for heart rate measurement. In this context, substantial research has been carried out, resulting in the registration of numerous inventions. Some of these notable inventions are listed below:

U.S. Pat. No. 10,945,614B2, issued on Mar. 16, 2021, by the United States Patent and Trademark Office (USPTO), describes a system and method for monitoring and treating cardiovascular diseases by determining heart rate (HR), respiration rate (RR), and classifying cardiac rhythms using atrial intracardiac electro gram (IEGM) and atrial pressure (AP) signals. The system includes an implantable device with a single lead featuring a pressure sensor and electrodes that communicate with a non-implantable device. The system processes IEGM and AP signals through spectrum transforms to obtain frequency spectra, identifying peaks to determine HR and RR. It distinguishes sinus rhythm from arrhythmias by analyzing the frequency power spectra and applying thresholds. The method includes steps for detecting atrial fibrillation (AF) and atrial flutter (AFI) by evaluating peaks within specific frequency ranges and their harmonics. The device also features a spectrum transform of IEGM and AP signals to assess cardiac rhythms accurately.

Chinese Patent No. CN105326491A, granted on May 22, 2018, describes a photo-electric reflection type pulse heart rate sensor that utilizes a self-adapting changeable threshold filter method to improve measurement accuracy by filtering out the interference of dicrotic waves. The method sets a threshold value at one-third of the peak voltage of the normal pulse wave. During sampling, values below this threshold are considered interference, while those above are considered part of the normal pulse wave. This adaptive approach adjusts the threshold dynamically based on each pulse's peak voltage, ensuring accurate heart rate measurement despite variations in pulse intervals. The method involves periodic sampling, comparing consecutive voltage values to identify the pulse ascent stage, recording peak voltages, and calculating the heart rate based on the time difference between successive peaks.

U.S. Pat. No. 10,335,045B2, issued on Mar. 16, 2021, in the United States, presents a novel method for remote heart rate (HR) estimation from facial video sequences under realistic conditions, addressing issues such as facial expressions and movement. The proposed solution utilizes a self-adaptive matrix completion (SAMC) algorithm to dynamically select reliable facial regions for HR measurement, thereby mitigating noise. The method involves tracking facial landmarks, warping the facial region of interest, and computing chrominance features from the RGB channels. SAMC leverages matrix completion theory to recover a low-rank feature matrix, enabling accurate HR estimation by filtering out noisy data. Evaluations on the MAHNOB-HCI (a multimodal dataset for emotion recognition) and MMSE-HR (a dataset containing heart rate data and RGB videos) datasets demonstrate the method's enhanced accuracy in short-term and long-term HR prediction, outperforming existing state-of-the-art techniques.

Chinese Patent No. CN112842312B, granted on Mar. 8, 2022, describes a heart rate sensor and a low-power-consumption self-adaptive heartbeat lock ring system utilizing photoplethysmography (PPG) technology. This system aims to reduce the power consumption of the LEDs used in heart rate monitoring by dynamically adjusting the duty cycle of the LED driver based on real-time heart rate changes. The invention includes a heart rate calculator module comprising a frequency-to-digital converter, divider, and digital filter. These components convert the PPG signal into digital form, filter high-frequency noise, and produce a digital heart rate signal. An adaptive window generator module, consisting of a heart rate differentiator, comparator, frequency divider, and window generator, further processes this signal to calculate heart rate change rates and generate self-adaptive window signals. The system adjusts the LED's on-time by determining the frequency division multiple, thereby reducing power consumption without compromising measurement accuracy. This adaptive adjustment ensures efficient LED operation, significantly lowering power usage compared to traditional methods.

Patent No. CN114469034B, issued on Jun. 30, 2023, in China, describes an abnormal heart rate monitoring method based on adaptive hybrid filtering, designed for noninvasive monitoring of various population groups, including those with heart disease history, “three-high” groups, high-intensity workers, and athletes. The system employs a combination of adaptive filtering techniques to reduce noise and improve accuracy in heart rate measurement across different states-rest, exercise, and sleep. It establishes characteristic models and heart rate thresholds for five user groups, dynamically adjusts filtering parameters, and provides real-time monitoring and early warning of abnormal heart rates through a Bluetooth-connected wearable device. This method significantly enhances the precision and reliability of heart rate monitoring, especially for populations with potential cardiovascular risks.

Patent No. CN106889980A, registered on Jun. 27, 2017, in China, details a self-adaptive switching heart rate detection method and device based on spectrogram technology for wearable heart rate monitoring. The method includes collecting photosignals from the skin surface using green, red, and infrared lights, and then applying Fourier transform to map these signals into a spectral intensity figure that shows the m-frequency relation over time. The system identifies the heart rate frequency peak region within this spectral intensity figure and assesses the continuity of the heart rate curve. If the green light signal becomes unclear or cannot accurately reflect heart rate changes, the device switches to a higher penetrability light, such as red or infrared, to maintain accurate detection. This switching mechanism, controlled by predefined criteria for signal clarity and frequency density, ensures continuous and precise heart rate monitoring across different conditions, including variations in skin tone and perspiration levels. The wearable device, which can be an intelligent bracelet, earphone, or watch, implements this method through functional modules programmed and stored in a computer-readable medium, offering real-time heart rate data with enhanced accuracy for users during various activities.

Patent No. CN117678998A, dated on Mar. 12, 2024, in China, outlines a non-contact heart rate detection method utilizing a self-adaptive projection plane and feature screening to extract pulse signals from face videos captured by a camera. This method employs photoplethysmography (PPG) to detect changes in blood volume via facial skin color variations due to heartbeat-induced blood flow changes. The process begins with face detection and tracking to isolate the region of interest (ROI), followed by spatial averaging to generate a three-dimensional RGB time-varying signal. The RGB signal undergoes dimensionality reduction using an advanced rPPG algorithm based on an adaptive projection plane, transforming it into a one-dimensional blood volume pulse (BVP) signal. This adaptive plane, derived through least squares fitting and orthogonal vector determination, adjusts to varying signal conditions, enhancing robustness against noise from lighting, facial movements, and expressions. The resultant BVP signal is then subjected to Butterworth band-pass filtering (0.7-4 Hz) to remove non-heartbeat-related noise. Heart rate calculation is performed by converting the filtered signal to the frequency domain using FFT to identify peak frequencies or applying a frequency tracking algorithm in the time-frequency domain to derive a time-varying heart rate sequence. This method ensures high signal-to-noise ratio and precise heart rate measurement under diverse real-world conditions.

Chinese Patent No. CN115719502A, granted on Feb. 28, 2023, presents a non-contact robust heart rate detection method adaptive to head rotation motion, designed for biomedical monitoring and computer vision. The method involves capturing face videos using imaging devices and detecting 68 facial feature points in real-time with CLNF (Constrained Local Neural Field). It extracts IPPG pulse wave signals from regions of interest (ROI) such as the cheeks and nose, known for rich capillary distribution, by calculating the gray average value of these regions. The original pulse signals are processed using trend-removing filtering and wavelet filtering algorithms to eliminate noise and identify the heart rate within a specific power spectrum range. The method further calculates the Euler angles of head posture to determine a novel signal quality index, which estimates adaptive noise covariance. A Kalman gain is adjusted by this covariance, forming a head rotation adaptive filter that dynamically filters motion artifacts based on rotation angles, ensuring accurate and robust heart rate estimation even during spontaneous head movements.

Patent No. CN113499049B, issued on Aug. 5, 2022, in China, presents a method for analyzing heart rate variability (HRV) data using self-adaptive multi-scale entropy (AMSE), enhancing the traditional Multiscale Sample Entropy (MSE) approach. The method employs Integral Mean Mode Decomposition (IMMD) to decompose HRV data into a series of multi-scale mean substitution datasets. These datasets undergo coarse granulation to establish self-adaptive scales. Subsequently, Sample Entropy (SampEn) values of these granulated datasets are computed to derive the AMSE, which accurately quantifies HRV complexity by adapting to the intrinsic dynamics of the data. This approach mitigates the limitations of fixed-scale MSE, offering precise assessment of nonlinear and non-stationary signals. The AMSE method's adaptability makes it particularly suitable for evaluating HRV under varying physiological and pathological states, thereby providing a robust tool for detailed cardiovascular regulation analysis and autonomic function assessment.

U.S. Patent No. U.S. Pat. No. 8,905,939B2, registered on Dec. 9, 2014, presents a method and apparatus for non-invasive estimation of cardiovascular parameters, specifically stroke volume (SV) and cardiac output (CO), utilizing arterial pulse pressure propagation time (t_prop). The method involves recording arterial pressure waveforms at multiple body locations, such as the aorta and radial artery, and measuring the transit time between these waveforms. This propagation time, indicative of arterial compliance, is integrated with higher order statistical moments (e.g., kurtosis, skewness) of the arterial pulse pressure waveform and patient-specific anthropometric data (e.g., age, height, weight, body surface area). These inputs are processed using a multivariate regression model to estimate SV and CO. The approach leverages the Bramwell-Hill equation to relate arterial compliance to pulse wave velocity, enhancing the accuracy and robustness of continuous hemodynamic monitoring, and minimizing the need for invasive calibration methods. The system includes modules for data acquisition, processing, and output, providing real-time cardiovascular assessment.

Patent No. US20220330842A1, registered on Oct. 20, 2022, in the United States, details a system for non-invasive blood pressure monitoring that utilizes an advanced method of calculating arterial pulse wave transit time (PWTT) adjusted for the pre-ejection period (PEP). The system employs a multi-sensor approach, incorporating electrocardiograph (ECG) sensors to detect electrical activity of the heart, acoustic sensors to monitor heart sounds and peripheral arterial pulses, optical sensors for plethysmographic data, and bioimpedance sensors to measure thoracic impedance changes. By capturing these diverse physiological signals, the system dynamically compensates for PEP, which represents the delay between electrical activation and mechanical ejection of blood from the ventricles. This compensation is crucial for deriving an accurate arterial PWTT. The system then analyzes changes in the compensated PWTT to trigger an occlusive blood pressure cuff when significant variations are detected, thereby ensuring precise and timely blood pressure measurements. Additionally, the system incorporates noise reduction techniques such as dynamic signal averaging and adaptive filtering, and it can calibrate PWTT measurements based on individualized patient-specific calibration factors, thereby enhancing measurement accuracy and reducing patient discomfort from frequent cuff inflations.

US Patent No. U.S. Pat. No. 11,793,460B2, dated Oct. 24, 2023, details an advanced, non-invasive sensor designed for continuous monitoring of cardiac output (CO), stroke volume (SV), thoracic fluid levels, electrocardiography (ECG) waveforms, heart (HR), rate arrhythmias, temperature, and motion/posture/activity levels in patients with congestive heart failure (CHF). This device, configured as a necklace, incorporates a sophisticated impedance cardiography system, ECG circuitry, and a tri-axis accelerometer, facilitating precise physiological measurements. Data is wirelessly transmitted to a patient's mobile device and subsequently relayed to a web-based platform for comprehensive analysis by healthcare providers. The system includes algorithms for signal processing and motion compensation, ensuring accurate readings during ambulatory activities. The design prioritizes patient comfort and compliance, featuring consistent electrode placement for reliable data acquisition. The device also enables real-time alerts for critical conditions such as fluid accumulation, thereby enhancing patient management through timely therapeutic adjustments.

Patent No. US20100113948A1, registered on May 6, 2010, in the United States, discloses a heart rate measurement system utilizing a reflective photoplethysmograph (PPG) sensor, optimized for continuous cardiovascular monitoring and designed to be discreetly worn behind the ear. The sensor employs light emitters and detectors to measure reflected light from the cranial surface of the auricula and the adjacent temporal scalp, capitalizing on regions with high vascularity and minimal skin pigmentation for superior signal quality. The system includes multiple detectors oriented at different angles to enhance anatomical compatibility and signal reliability. A sophisticated data processor executes algorithms for real-time heart rate calculation, leveraging frequency spectrum analysis and motion compensation techniques using ambient light measurements during emitter-off periods. Additionally, the system integrates an accelerometer for artifact correction and activity recognition. This configuration, featuring low-power consumption, wireless data transmission, and robust artifact compensation, facilitates continuous, non-invasive cardiovascular monitoring in pervasive healthcare applications.

patent No. U.S. Pat. No. 8,666,482B2, issued on Mar. 4, 2014, in the United States, presents an advanced method and system for HRV measurement, employing sensors such as ECG straps and blood pressure monitors. It integrates controlled breathing protocols and sophisticated algorithms for real-time processing and exclusion of irregular R-R intervals. The system enables precise HRV calculation using both time and frequency domain analyses, offering continuous and short-term measurement modes. It provides real-time feedback, training recommendations, and alerts for overtraining and heart failure management. Additionally, it incorporates features for mood tracking, fitness evaluation, and centralized data collection, thereby facilitating comprehensive, non-invasive monitoring and personalized management of cardiovascular and autonomic nervous system health.

Recent studies have demonstrated that the rhythmic contraction and expansion of blood vessels during each heartbeat induce subtle changes in skin color, which are imperceptible to the human eye but can be readily detected by computer vision. This allows recorded facial videos to be effectively used for heart rate (HR) estimation.

One of the emerging methods for HR estimation involves using image processing techniques to analyze facial videos. Subtle changes in skin color, caused by blood flow associated with heart rate, can be detected through methods such as pixel analysis or feature extraction. Advanced technologies for estimating HR from facial videos utilize color spectrum analysis or machine learning algorithms like neural networks to identify specific patterns related to HR changes. These patterns may include variations in skin color, facial movements, or light fluctuations.

Despite significant progress in recent years, estimating HR from facial videos still faces several challenges. Key issues include the impact of environmental factors such as head movements, facial expressions, and variable lighting, which can cause skin color changes that may be mistaken for blood flow variations. Many existing methods perform well under ideal conditions but suffer from decreased accuracy in real-world scenarios with low light or high noise. Additionally, the datasets used to train machine learning algorithms often lack diversity in terms of subjects, ages, races, and lighting conditions, further limiting their generalizability.

Therefore, the present invention addresses the limitations of previous methods by introducing an innovative approach that enhances both usability and accuracy. The proposed technology aims to overcome these challenges, providing a more reliable and effective solution.

### DESCRIPTION OF THE INVENTION

The invention introduces a system for estimating heart rate from facial images utilizing advanced image processing, machine vision, and noise removal techniques. The proposed system employs a high-quality camera to capture video footage of a person's face. Through sophisticated image processing and machine vision methods, the system monitors and analyzes the subtle color changes in the facial skin caused by the contraction and expansion of blood vessels with each heartbeat. To enhance the accuracy of heart rate estimation, the system applies the self-adaptive matrix technique to effectively remove image noise.

In this method, high-quality cameras installed in the environment are used to film the subject. Data collection is a crucial step in the process of heart rate estimation from facial images, which involves image processing and noise removal using the self-adaptive matrix technique. This step encompasses optimizing camera settings, lighting conditions, and video frame rate. For accurate and detailed data collection, it is essential to use a high-resolution camera, such as HD or 4K, to provide clear and detailed images. Furthermore, a high frame rate (at least 30 frames per second or higher) is vital to capture the rapid changes in skin color caused by blood flow and heart rate.

The lighting in the environment where the camera is installed is also critical. It is important to use natural light or consistent ambient lighting to avoid shadows and unwanted variations in image brightness. However, direct and strong light can cause reflections and unrealistic changes in the image, so diffused and indirect lighting is preferable. Additionally, the camera should remain fixed and motionless, ensuring that any changes in the image are solely due to physiological variations.

The video recording duration should be sufficient to capture multiple heartbeat cycles. The camera must be positioned at an angle that fully and clearly displays the subject's face, with the entire face fitting within the frame. Additionally, it is beneficial to record the video from various angles (e.g., frontal, profile) and under different physiological conditions (e.g., at rest, during physical activity, under stress) to evaluate the impact of these factors on the accuracy of heart rate estimation.

After collecting the image data, pre-processing is conducted to enhance image quality, reduce noise, and prepare the data for subsequent steps. This stage involves several tasks, including image stabilization, brightness and contrast adjustment, noise reduction, identification and extraction of the desired area, and extraction of color features.

Video images can exhibit motion noise due to camera shake or head movement. To mitigate these issues, image stabilization techniques, such as Optical Flow algorithms or the camera's internal stabilization capabilities, are employed. Algorithms like Lucas-Kanade Optical Flow are also used to stabilize video frames, reducing the impact of motion-induced changes. Additionally, variations in image brightness over time can adversely affect processing accuracy. To address this, techniques such as Histogram Equalization or Adaptive Histogram Equalization are applied to balance and adjust brightness, ensuring consistent image quality.

A video recording of a person's face comprises numerous frames, with each frame being a still image captured at a specific moment. Each frame is subsequently divided into a grid of pixels for further analysis.

In the next step, as illustrated in the flowchart (FIG. 1, No. 1) time series of video frames is captured from the subject's face at successive intervals to obtain image sequences (FIG. 4). A convolutional neural network (CNN) is then employed (FIGS. 3 and 4, No. 2 and 3) to detect the edges of the images, identifying and tracking the range and movements of the head. The predetermined points on the forehead and cheeks, which are optimal for detection due to the high concentration of blood vessels, are evaluated. If excessive noise is present in these areas due to significant head movements or changing light conditions, and the image cannot be adequately reconstructed using standard noise removal methods, the process advances to the second phase to identify the most reliable point for analysis.

First, a mask pattern is created within the database (FIG. 5), consisting of A areas. Each area is assigned a unique ID and encompasses a specific number of pixels from the image. This mask is then applied to each frame obtained from the video of the subject's face, ensuring alignment with the corresponding facial regions in every frame (FIGS. 1 and 5, No. 4).

To assess pixel similarity within each mask area, the Pearson correlation coefficient is employed (FIG. 1, No. 5). This approach identifies the most reliable points in the image, which are the points that remain stable over time and can be effectively utilized for motion tracking and other image processing tasks.

In this method, the correlation coefficient is calculated separately for each area of the mask, with each area assigned a unique ID, resulting in A correlation coefficients per frame. This procedure is performed for all frames. After computing the correlation coefficients for each frame, the system identifies k IDs, where k is proportionate to A, with coefficients closest to one, representing the most reliable regions for that frame (FIG. 1, No. 7). These selected k IDs for each frame are then stored in memory.

After calculating the correlation coefficients for all frames, the control unit examines the stored k IDs for each frame. It identifies the IDs that appear consistently across all frames or have the highest frequency of occurrence. These IDs represent the most reliable areas, indicating regions with the least variation over time.

In the next step, the control unit selects the single most reliable area from the identified regions. To achieve this, it sums the correlation coefficients for each of the k IDs across all n frames (FIG. 1, No. 8). The area with the highest cumulative correlation coefficient is deemed the most consistent throughout the frames and is chosen as the best reliable region for further processing (FIG. 1, No. 8).

As we have obtained the most reliable area of the frames, we now need to identify the best time interval during which the RGB values of the selected area exhibit the least changes (FIG. 1, No. 6). To do so, we employ a structured and systematic approach involving image processing and statistical analysis. Initially, we select a frame of interest and calculate the difference in RGB values between each pixel in consecutive frames, resulting in a matrix which is called a difference matrix. In the obtained matrix, rows represent the frames and columns represent individual pixels.

For each pair of consecutive frames Fᵢ and Fᵢ₊₁, where i ranges from 1 to n−1 (with n being the total number of frames), we compute the differences in RGB values for each pixel. These differences are stored in a difference matrix D, where D [i] [p] denotes the difference for pixel p between frames Fᵢ and Fᵢ₊₁. Each row of the matrix encapsulates the RGB differences for a specific frame pair across all pixels, which is obtained using the Euclidean distance formula (FIG. 9).

Where p and i are number of pixels and frames, respectively.

Next, we sum the differences for each row to derive a single representative value for the variation between each frame pair. This sum is calculated in the formula (FIG. 10).

Wherein m is the total number of pixels in each frame. This process yields a sequence of sums, each corresponding to the overall variation between successive frames.

To accurately determine the time interval with minimal pixel changes, the sums obtained from Equation 2 are evaluated based on the following two conditions:
1. The sums should be as small as possible.
2. The sequence of these minimum sums should be as long as possible.

By applying these two conditions, we ensure that the maximum number of consecutive frames with the least pixel changes relative to each other is identified.

Once the optimal time frame and the most reliable area of the image have been identified, all subsequent processing and calculations will be confined to this time interval and within the selected region. This targeted approach ensures that the data used for analysis is derived from the most stable and consistent part of the video, thereby enhancing the accuracy and reliability of the results.

In the next step, the system calculates the R, G, and B values for the pixels within the determined area. These values are then passed through a skin color detection filter (Table 1) to identify and exclude areas that are not recognized as skin color (refer to FIG. 1, No. 9).

According to KOVAC's guidelines, pixels are identified as skin color if they fall within the range (R>G & R>B & B<200). Pixels outside this range are classified as noise.

In the subsequent phase, the chrominance (color characteristic) of all pixels within the identified reliable area is calculated for each frame within the previously determined time interval (FIG. 1, No. 11). Chrominance (C) is derived from the normalized values of the primary colors R, G, and B, using the formula (FIG. 11).

wherein a, B, and λ are coefficients representing the contribution of each color channel to the chrominance value.

Assuming the number of pixels in the selected area is m and the number of frames in the determined interval is n, the resulting chrominance values will form an n×m matrix. These chrominance values, displayed in a matrix format (FIG. 1, No. 12), represent the chrominance of each pixel across different frames. To enhance accuracy and eliminate potential noise in the chrominance matrix, which could result from changes in facial expressions or discrepancies in the skin color filter, we scrutinize the columns of this matrix. Each column represents the chrominance values of a pixel across different frames. By identifying and analyzing columns where the chrominance values significantly deviate from the norm, we can determine and isolate areas of high variability and potential noise.

Ultimately, self-adaptive matrix techniques are employed to eliminate noise and filter out unwanted signals (FIG. 1, No. 12). This process involves completing the specified areas in the chrominance matrix using self-adaptive matrix methods. The matrix continuously analyzes incoming signals, removing noise while enhancing useful signals. This approach ensures the acquisition of more accurate and high-quality signals for heart rate estimation.

The matrix contains weights that adapt to changes in input signals. By applying adaptive filters based on these matrix weights, detected noise is effectively removed. These weights are updated in real-time based on changes in the input signals, ensuring optimal matching and noise removal. Subsequently, the self-adaptive matrix amplifies the useful signals related to heart rate using statistical analysis and optimization algorithms. Initially, several columns are selected as reference points, and the values identified as noise in each column are marked accordingly. This optimized algorithm allows for precise noise identification and signal amplification, leading to improved heart rate estimation accuracy.

After completing this step, we obtain a chrominance matrix with normalized values and no noise (FIG. 1, No. 13). This matrix can be used to plot a graph. By summing the values of each row in the matrix, which represent the expansion or contraction of the heart at that specific time (frame), we form a new matrix of dimensions n×1 (FIG. 1, No. 14). Plotting this matrix yields an approximately sinusoidal waveform. The greater the number of frames in the video, the higher the accuracy of the resulting waveform, making it more closely resembles a true sinusoid (FIG. 1, No. 15).

In the final step, the number of waveform peaks within the measured time interval is counted. This count is then extrapolated to determine the number of waveforms occurring in one minute, thereby estimating the heart rate (HR) (FIG. 1, No. 16).

For illustrative purposes, let's consider matrix A [8×6] as our chrominance matrix, representing 6 pixels across 8 frames (FIG. 12). (Note: All values in this example are hypothetical and intended to clarify the process).

After computing the chrominance values, the columns of the matrix representing the chrominance values for each pixel across different frames are evaluated. Arrays with values that deviate significantly by a certain percentage from other arrays are identified as noise and are subsequently set to zero (FIG. 13).

In the subsequent step, the self-adaptive matrix technique is utilized to restore and complete the arrays that were previously zeroed out (FIG. 14).

In the next step, the values of the arrays in each row, which represent the heart's expansion or contraction at that specific time (frame), are summed together (FIG. 15).

Finally, by plotting the graph of the matrix, which closely resembles a sinusoidal waveform, we can accurately estimate the heart rate (HR) (FIG. 8.)

### BRIEF DESCRIPTION OF FIGURES

FIG. 1: The block diagram illustrating the steps of the invention is displayed.

FIG. 2: A photograph of the subject taken in a public setting.

FIG. 3: Illustration of range detection and head movement tracking utilizing a convolutional neural network.

FIG. 4: A time series of video frames capturing the subject's face at successive moments is depicted.

FIG. 5: Illustration of Mask Placement on the Face

FIG. 6: Display of the reliable areas identified by the control unit.

FIG. 7: Illustration of the selected area from the identified reliable regions.

FIG. 8: Illustration of the final graph resulting from expansion and contraction of the heart in different frames.

FIG. 9: Euclidean distance formula

FIG. 10: The formula for detecting the amount of changes in both pairs of frames

FIG. 11: Chrominance Matrix

FIG. 12: Example of Chrominance Matrix FIG. 13: Matrix for detection of skin discoloration

FIG. 14: Example of filling the skin discoloration areas by self-adaptive Matrix

FIG. 15: Matrix showing procedure of the expansion and contraction of the heart

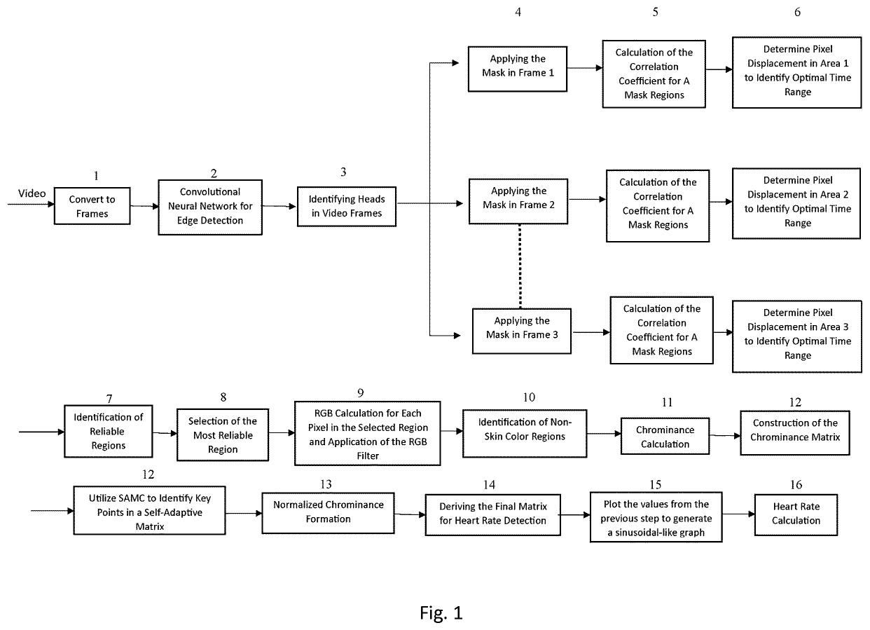

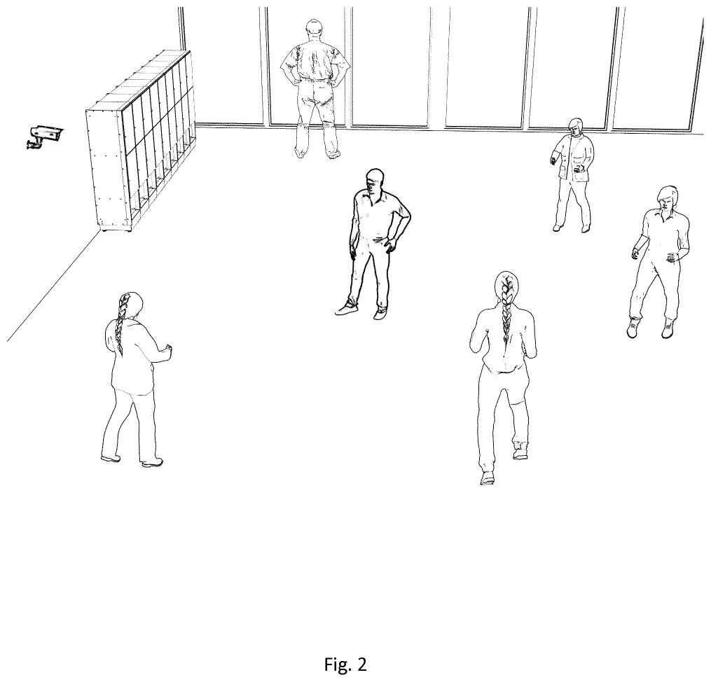

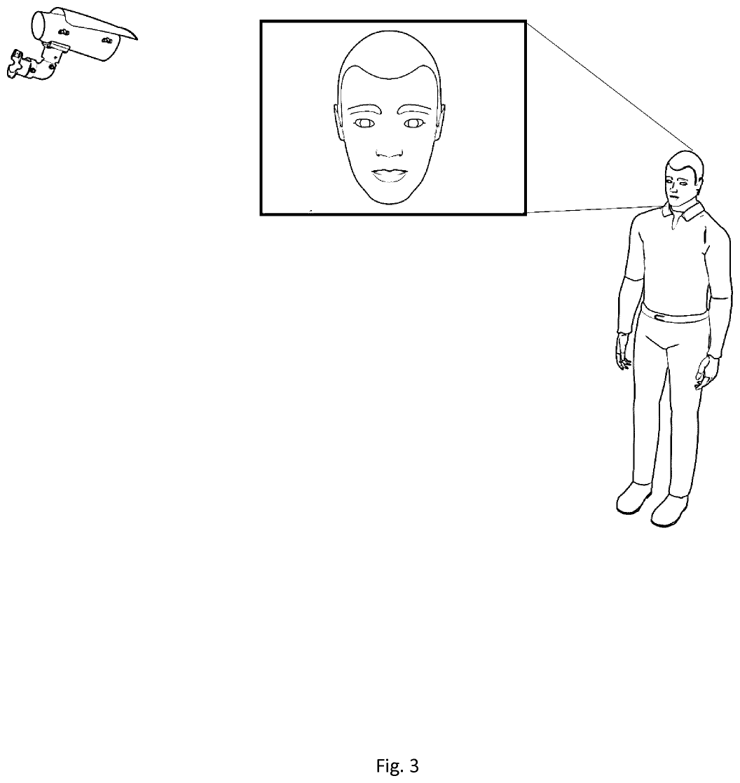

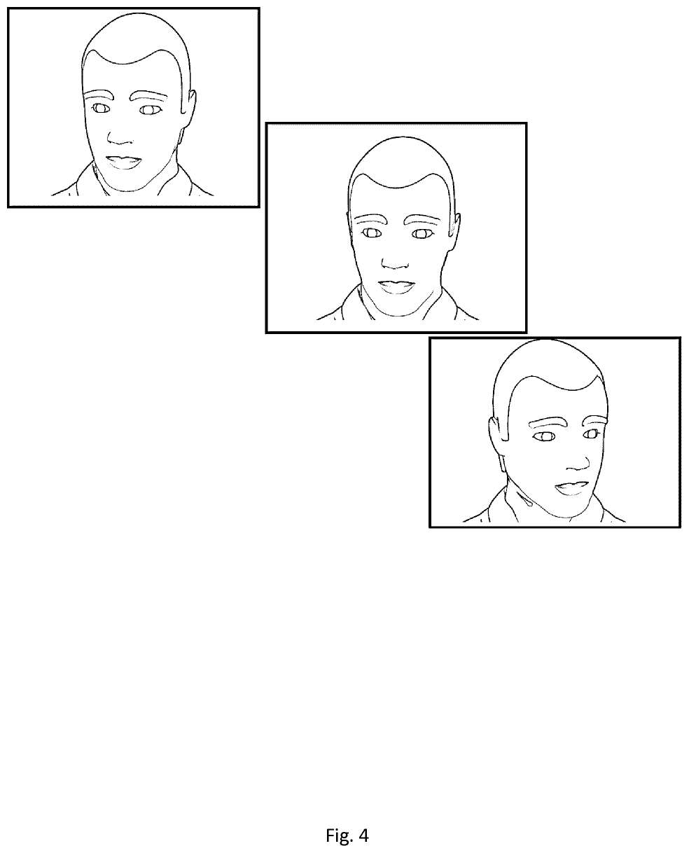

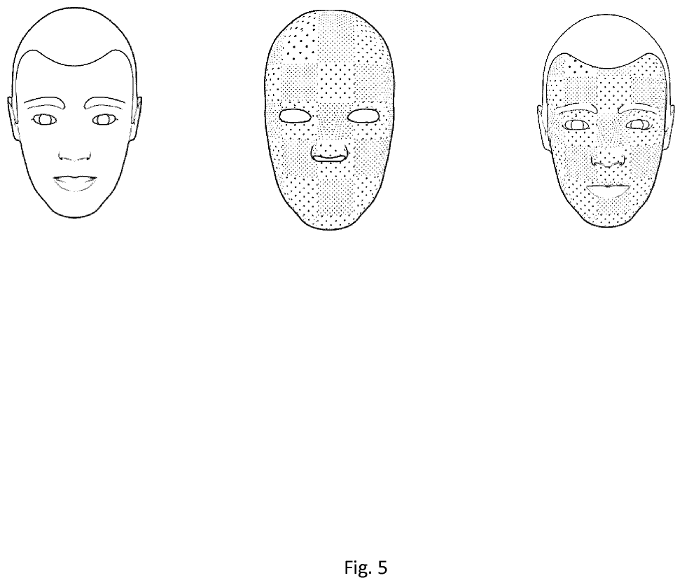

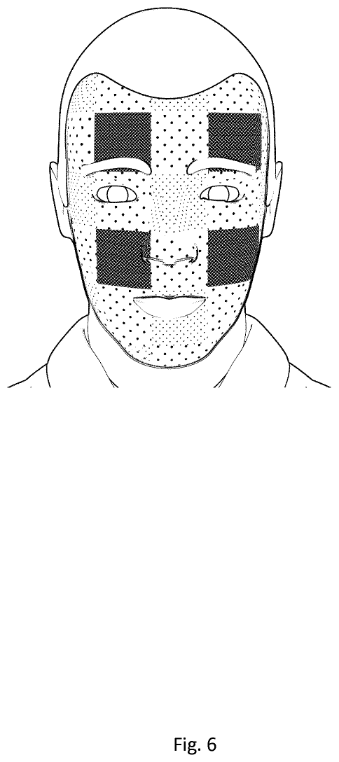

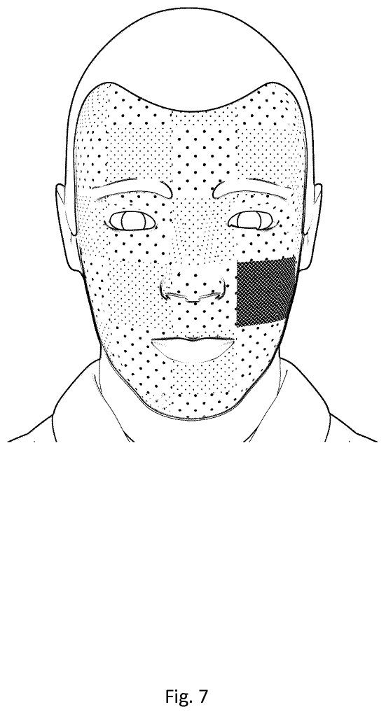

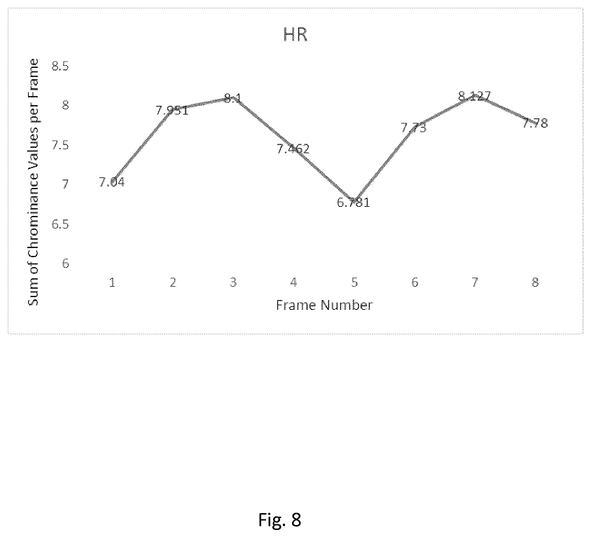

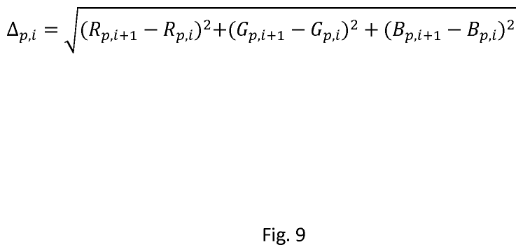

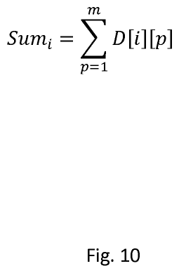

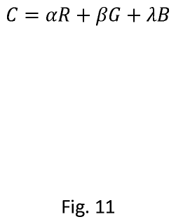

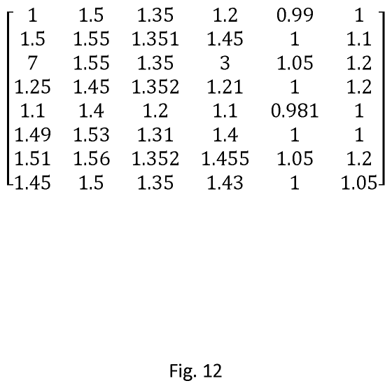

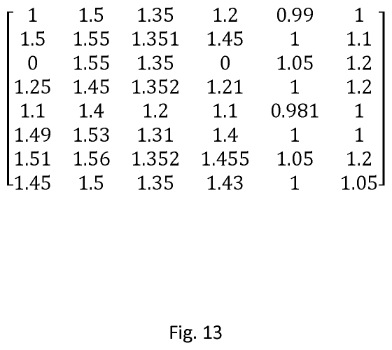

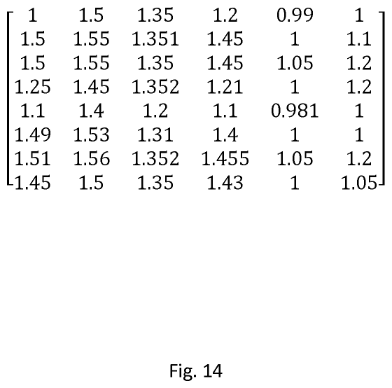

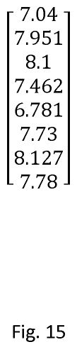

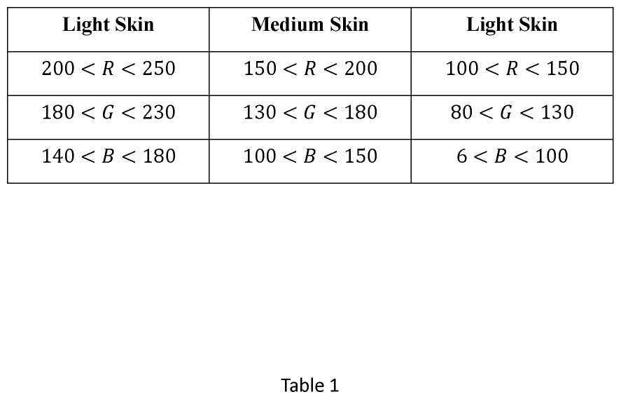

Table 1: Skin color detection filter

What is claimed is:

1. The invention of heart rate estimation from facial images using image processing and self-adaptive matrix noise removal that is equipped with image processing algorithms and machine vision and various electronic equipment including at least one high-quality camera for video recording and at least one control unit that includes at least one storage unit and at least one database and at least one template for the mask and at least one filter.

2. The system and structure of claim 1, wherein, a high-resolution camera is employed to capture images of the subject.

3. The system and structure of claim 1, wherein a convolutional neural network is employed to detect distance ranges and to identify and track head movements.

4. The system and structure of claim 1, wherein specific regions of the forehead and cheeks are selected for their high density of blood vessels, rendering them ideal for diagnostic purposes.

5. The system and structure of claim 1, wherein, if the predetermined areas are deemed unsuitable, the process will advance to the secondary detection phase.

6. The system and structure of claim 1, wherein the mask comprises A distinct regions, each designated with a unique identifier.

7. The system and structure of claim 1, wherein the mask is applied to the subject's face image in each frame of the video captured from the subject's face.

8. The system and structure of claim 1, wherein the degree of similarity of pixels within each area of the mask in each frame is determined using the Pearson correlation coefficient which this mathematical operation of correlation is employed to identify the most reliable areas within the image.

9. The system and structure of claim 1, wherein k regions, each having a correlation coefficient of one or the highest possible value, are identified as the optimal regions for each frame and stored in memory.

10. The system and structure described in claim 1 involve a process where the control unit analyzes the stored ID numbers for each frame and it identifies IDs that are either repeated across all frames or have the highest frequency of occurrence, and designates these IDs as reliable areas.

11. The system and structure of claim 1, wherein the control unit aggregates the correlation coefficient values for each selected area, computed for each frame, and identifies the area with the highest cumulative correlation coefficient as the most reliable area.

12. The system and structure of claim 1, wherein the optimal time frame for the film is determined by deriving the correlation coefficient from the correlation coefficients of selected areas within each frame, thereby minimizing abrupt changes.

13. The system and structure of claim 1, wherein the control unit computes the red (R), green (G), and blue (B) values of the pixels within the specified area and applies these values to a skin color detection filter for processing.

14. The system and structure of claim 1, wherein the chrominance values for all pixels within the reliable area are computed for each frame within the designated time interval as determined from the preceding step.

15. The system and structure as described in claim 1 involves creating a chrominance matrix of dimensions [n×m], where ‘m’ denotes the number of pixels within the selected area and ‘n’ represents the number of frames within the specified interval.

16. The system and structure as described in claim 1, wherein the RGB color range of pixels detected outside the skin color spectrum is defined within the columns of the chrominance matrix.

17. The system and structure of claim 1, wherein self-adaptive matrix techniques are utilized for the purposes of noise reduction and filtration.

18. The system and structure of claim 1, wherein the values within each row of the matrix, which correspond to the heart's expansion or contraction at each time frame, are aggregated that these aggregated values produce a new matrix with dimensions [n×1].

19. The system and structure as described in claim 1, wherein plotting the matrix graph of [n×1] yields an approximately sinusoidal waveform and by counting the peaks within the waveform over a specified time interval, the frequency of these waveforms can be determined per minute, allowing for the estimation of the heart rate (HR).
